(19) United States
(12) Patent Application Publication
(10) Pub. No.: US 2022/0246300 A1
(43) Pub. Date: Aug. 4, 2022

(54) DIAGNOSIS ASSISTANCE APPARATUS AND DIAGNOSIS ASSISTANCE SYSTEM

(71) Applicant: CANON MEDICAL SYSTEMS CORPORATION, Otawara-shi (JP)

(72) Inventors: Yusuke KANO, Nasushiobara (JP); Kazumasa NORO, Shioya-gun (JP)

(73) Assignee: CANON MEDICAL SYSTEMS CORPORATION, Otawara-shi (JP)

(21) Appl. No.: 17/591,083

(22) Filed: Feb. 2, 2022

(30) Foreign Application Priority Data
Feb. 3, 2021 (JP) 2021-015952

(51) Int. Cl. G16H 50/20; G06F 40/30; G06F 40/279; G06F 3/14; G06F 3/0482

(52) U.S. Cl. CPC G16H 50/20; G06F 40/30; G06F 3/0482; G06F 3/14; G06F 40/279

(57) ABSTRACT

A diagnosis assistance apparatus according to an embodiment includes processing circuitry. The processing circuitry obtains text information representing each of a plurality of diagnosis/treatment records at a plurality of points in time, as diagnosis/treatment data related to a first patient. The processing circuitry performs a natural language processing process to extract a predetermined word from the text information, classifies each of the plurality of diagnosis/treatment records into at least one category in accordance with the extracted word, and calculates breakdown information indicating frequency of appearance of one or more categories related to one or more diagnosis/treatment records in a predetermined period of time among the plurality of diagnosis/treatment records. The processing circuitry causes a display to display a display screen including a temporal transition and an accumulated total related to the frequency of appearance of the one or more categories on the basis of the calculated breakdown information.

### CROSS-REFERENCE TO RELATED APPLICATIONS

This application is based upon and claims the benefit of priority from Japanese Patent Application No. 2021-015952, filed on Feb. 3, 2021; the entire contents of which are incorporated herein by reference.

### FIELD

Embodiments described herein relate generally to a diagnosis assistance apparatus and a diagnosis assistance system.

### BACKGROUND

Conventionally, a technique is known by which various types of diagnosis/treatment data needed by a medical doctor to analyze a diagnosis or a treatment plan are displayed on a single screen. The medical doctor performs a diagnosing process or treatment for a patient by comprehensively looking at the various types of diagnosis/treatment data displayed on the screen.

In relation to this, there are situations in which medical doctors determine a patient for whom observation or intervention is to be implemented with priority, by checking chronological changes in conditions of patients, on the basis of not only numerical value information such as vital signs but also what is written in diagnosis/treatment records that are kept as medical records. For this reason, there is a demand for making it possible to easily understand chronological changes in the conditions of patients on the basis of what is written in diagnosis/treatment records.

### BRIEF DESCRIPTION OF THE DRAWINGS

FIG. 1 is a diagram illustrating an exemplary configuration of a diagnosis assistance system according to an embodiment;

FIG. 2 is a diagram illustrating an exemplary configuration of a diagnosis assistance apparatus according to the embodiment;

FIG. 3 is a drawing illustrating an example of diagnosis/treatment data according to the embodiment;

FIG. 4 is a diagram illustrating examples of categories according to the embodiment;

FIG. 5 is a diagram for explaining a classification of diagnosis/treatment record entries of diagnosis/treatment records according to the embodiment;

FIG. 6 is a flowchart illustrating an example of a diagnosis assisting process according to the embodiment; and

FIG. 7 is a drawing illustrating an example of a display screen displayed by the diagnosis assistance system according to the embodiment.

### DETAILED DESCRIPTION

A diagnosis assistance apparatus according to an embodiment includes processing circuitry. The processing circuitry is configured to obtain text information representing each of a plurality of diagnosis/treatment records at a plurality of points in time, as diagnosis/treatment data related to a first patient. The processing circuitry is configured to perform a natural language processing process to extract a predetermined word from the text information, to classify each of the plurality of diagnosis/treatment records into at least one category in accordance with the extracted word, and to calculate breakdown information indicating frequency of appearance of one or more categories related to one or more diagnosis/treatment records in a predetermined period of time among the plurality of diagnosis/treatment records. The processing circuitry is configured to cause a display to display a display screen including a temporal transition and an accumulated total related to the frequency of appearance of the one or more categories on a basis of the calculated breakdown information.

Exemplary embodiments of a medical image diagnosis apparatus and a medical information display controlling device will be explained, with reference to the accompanying drawings. In the description presented below, some of the constituent elements having the same or substantially the same functions as those described with reference to already-explained drawings will be referred to by using the same reference characters, and duplicate explanations are provided only when necessary. Further, mutually the same elements in different drawings may be depicted in different sizes or in different scales. Further, from the viewpoint of ensuring visibility of the drawings, for example, the reference characters in the description of the drawings may be provided only for principal constituent elements. Certain constituent elements having the same or substantially the same functions may not be provided with reference characters, in some situations.

A technique is known by which various types of diagnosis/treatment data necessary for a medical doctor when analyzing a diagnosis or a treatment plan are displayed on a single screen. The medical doctor performs a diagnosing process or treatment for a patient by comprehensively looking at the various types of diagnosis/treatment data displayed on the screen.

In relation to this, there are situations in which medical doctors determine a patient for whom observation or intervention is to be implemented with priority, by checking chronological changes in conditions of patients, on the basis of not only numerical value information such as vital signs but also what is written in diagnosis/treatment records that are kept as electronic medical records. For this reason, there is a demand for making it possible to easily understand chronological changes in the conditions of patients on the basis of what is written in diagnosis/treatment records.

For example, a technique is known by which text information input in an electronic medical record is summarized. However, when what is written is summarized, although it becomes easy to understand the outline, it is impossible to grasp temporal changes even from time-series information such as that in electronic medical records. As another example, a technique is known by which the degree of a symptom of a patient is obtained, on the basis of frequency of appearance and timing thereof with respect to a word included in text information. However, conditions of patients cannot necessarily be expressed as chronological changes of a specific symptom. In some situations, mutually-different types of symptoms may be described, or patients' conditions may be described not only with symptoms, but by using a different form such as an observation on his/her body. For this reason, there are some situations in which the frequency of appearance of a word in the text information may not match the degree of the patient's condition which the medical doctor wishes to understand. Consequently, it has been difficult to easily understand temporal changes in patients' states, on the basis of what is written in diagnosis/treatment records.

To cope with this situation, disclosed in the present embodiments is a diagnosis assistance apparatus 10 and a diagnosis assistance system 1 capable of performing a diagnosis assisting process to visualize chronological changes in a patient's state, on the basis of text information from a diagnosis/treatment record.

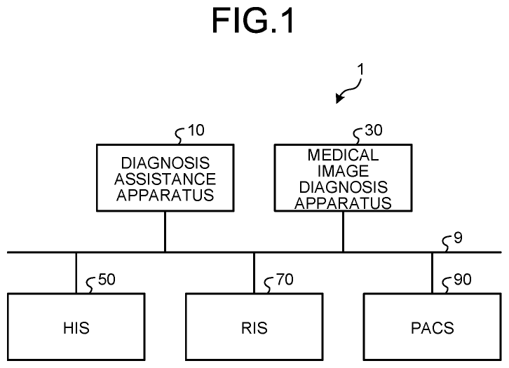

FIG. 1 is a diagram illustrating an exemplary configuration of the diagnosis assistance system 1 according to an embodiment. As illustrated in FIG. 1, the diagnosis assistance system 1 includes the diagnosis assistance apparatus 10, a medical image diagnosis apparatus 30, a Hospital Information System (HIS) 50, a Radiology Information System (RIS) 70, and a medical image management system (a Picture Archiving and Communication System, PACS) 90. The apparatuses and devices in the diagnosis assistance system 1 are installed in a hospital, for example, and are capable of communicating with other devices and apparatuses via a network 9 such as an intra-hospital Local Area Network (LAN). In this situation, the HIS 50 may be connected to an external network, in addition to the intra-hospital LAN.

The HIS 50 is a system configured to manage information occurring in the hospital. The information occurring in the hospital includes information such as patient information and examination order information. Each of the record entries included in the patient information has, as items thereof, a patient ID, the patient's name (a surname and a given name), the age (the date of birth), the gender, the height, the weight, the blood type, and the like. Each of the record entries included in the examination order information has, as items thereof, an examination ID capable of identifying a medical examination (hereinafter, “examination”), a patient ID, information indicating inpatient or outpatient, an examination code, the diagnosis/treatment department, the type of the examination, an examined site of the body, a scheduled date/time of the examination, and the like.

The examination ID is issued when the examination order information is input and is, for example, an identifier for uniquely identifying the examination order information in the one hospital. The patient ID is an identifier assigned to each patient and is used for uniquely identifying the patient in the one hospital, for example. The examination code is defined in the one hospital and is an identifier for uniquely identifying an examination. The diagnosis/treatment department indicates a specialty field category of the diagnosis/treatment in medicine, for example. More specifically, the diagnosis/treatment department may be internal medicine, surgery, or the like. The type of the examination indicates an examination using one or more medical images. For instance, examples of the type of the examination include an X-ray examination, a Computed Tomography (CT) examination, and a Magnetic Resonance Imaging (MRI) examination. Examples of the examined site include the brain, the kidneys, the lungs, and the liver.

When the examination order information is input by an examination requesting doctor, for example, the HIS 50 is configured to transmit, to the RIS, the input examination order information and the patient information identified by the examination order information. Further, in this situation, the HIS 50 is configured to transmit the patient information to the PACS.

The RIS 70 is a system configured to manage examination appointment information related to radiation examination work. For example, the RIS 70 is configured to receive the examination order information transmitted from the HIS 50, to aggregate received examination order information after appending various types of setting information thereto, and to manage the aggregated information as the examination appointment information. More specifically, upon receipt of the patient information and the examination order information transmitted from the HIS 50, the RIS 70 is configured to generate the examination appointment information necessary for causing the medical image diagnosis apparatus 30 to operate, on the basis of the patient information and the examination order information that were received. The examination appointment information includes, for example, information required to perform the examination, such as the examination ID, the patient ID, the type of the examination, and the examined site of the body. The RIS 70 is configured to transmit the generated examination appointment information to the medical image diagnosis apparatus 30.

The medical image diagnosis apparatus 30 is an apparatus configured to generate medical image data on the basis of data acquired from an examined subject. As the medical image diagnosis apparatus 30, it is possible to use, as appropriate, any of various types of medical image diagnosis apparatuses such as an X-ray diagnosis apparatus, an X-ray Computed Tomography (CT) apparatus, a Magnetic Resonance Imaging (MRI) apparatus, an ultrasound diagnosis apparatus, a Single Photon Emission Computed Tomography (SPECT) apparatus, a Positron Emission computed Tomography (PET) apparatus, a SPECT-CT apparatus in which a SPECT apparatus and an X-ray CT apparatus are integrated together, or a PET-CT apparatus in which a PET apparatus and an X-ray CT apparatus are integrated together.

The medical image diagnosis apparatus 30 is configured to perform the examination, on the basis of the examination appointment information transmitted from the RIS 70, for example. The medical image diagnosis apparatus 30 is configured to generate and transmit, to the RIS 70, examination execution information indicating execution of the examination. In this situation, the RIS 70 is configured to receive the examination execution information from the medical image diagnosis apparatus 30 and to output the received examination execution information to the HIS 50 or the like, as the most up-to-date examination execution information. For example, the HIS 50 is configured to receive the most up-to-date examination execution information and to manage the received examination execution information. The examination execution information includes the examination appointment information such as the examination ID, the patient ID, the type of the examination, and the examined site of the body, as well as an execution date/time of the examination.

The medical image diagnosis apparatus 30 is configured to convert the generated image data into a format compliant with a Digital imaging and Communication in Medicine (DICOM) standard, for example. In other words, the medical image diagnosis apparatus 30 is configured to generate medical image data to which a DICOM tag is appended as additional information.

The additional information includes, for example, the patient ID, the examination ID, an apparatus ID, and an image series ID and is standardized according to the DICOM standard. The apparatus ID is information used for identifying the medical image diagnosis apparatus 30. The image series ID is information used for identifying one session of imaging process performed by the medical image diagnosis apparatus 30 and may include, for example, the body site of the examined subject (patient) being imaged, a time at which an image was generated, a slice thickness, and a slice position. For example, as a result of performing a CT examination or an MRI examination, a tomographic image in each of a plurality of slice positions is obtained as medical image data.

The medical image diagnosis apparatus 30 is configured to transmit the generated medical image data to the PACS 90. The PACS 90 is a system configured to manage various types of medical image data.

For example, the PACS 90 is configured to receive the patient information transmitted from the HIS 50 and to manage the received patient information. The PACS 90 includes a memory for managing the patient information. For example, the PACS 90 is configured to receive the medical image data transmitted from the medical image diagnosis apparatus 30 and to store the received medical image data into the memory thereof so as to be kept in correspondence with the patient information. In this situation, to the medical image data saved in the PACS 90, the additional information such as the patient ID, the examination ID, the apparatus ID, the image series ID, and the like are appended. Accordingly, by conducting a search using a patient ID or the like, an operator is able to obtain necessary patient information from the PACS 90. Further, by conducting a search using a patient ID, an examination ID, an apparatus ID, an image series ID, and/or the like, the operator is able to obtain necessary medical image data from the PACS 90.

In this situation, the HIS 50 is configured to receive an electronic medical record generated by a clinical doctor being the examination requesting doctor, for example, and the examination execution information corresponding to the electronic medical record and to further store, into the memory thereof, the received electronic medical record and examination execution information so as to be kept in correspondence with each other. In this situation, as explained above, because the examination execution information includes the examination ID, the patient ID, the type of the examination, the examined site of the body, and the execution date/time of the examination, the operator is able to obtain a necessary electronic medical record from the HIS 50, by conducting a search using a patient ID, an examination ID, and/or the like. Although the electronic medical record is stored in the memory of the HIS 50 in the present embodiment, the electronic medical record may be stored in a memory of any other apparatus or device in the diagnosis assistance system 1, as long as it is possible to conduct the search while using one or more IDs.

Further, the RIS 70 is configured to receive, for example, an image interpretation report generated in accordance with an input from an image interpreting doctor and the examination execution information corresponding to the image interpretation report and to further store, into the memory thereof, the received image interpretation and examination execution information so as to be kept in correspondence with each other. In this situation, as explained above, because the examination execution information includes the examination ID, the patient ID, the type of the examination, the examined site of the body, and the execution date/time of the examination, the operator is able to obtain a necessary image interpretation report from the RIS 70 by conducting a search using a patient ID, an examination ID, and/or the like. Although the image interpretation report is stored in the memory of the RIS 70 in the present embodiment, the image interpretation report may be stored in a memory of any other apparatus or device in the diagnosis assistance system 1, as long as it is possible to conduct the search while using one or more IDs.

The diagnosis assistance apparatus 10 is configured to perform the diagnosis assisting process. The diagnosis assistance apparatus 10 is configured to obtain, via the network 9, various types of diagnosis/treatment data from the medical image diagnosis apparatus 30, the HIS 50, the RIS 70, and the PACS 90 and to perform various types of information processing processes by using the obtained diagnosis/treatment data. For example, the diagnosis assistance apparatus 10 is realized by using a computer such as a workstation that includes a processor and memory elements such as a ROM and a RAM as hardware resources. For example, the diagnosis assistance apparatus 10 has an integrated viewer installed therein, for example. The integrated viewer is an application configured to present medical information to a user in an integrated manner. The integrated viewer may adopt any installation mode such as that of a web application, a FAT client application, or a thin client application.

The diagnosis/treatment data is information indicating diagnosis/treatment records which medical workers were able to learn about a physical status, the condition of a disease, treatment for the patient, and the like, in the process of diagnosis/treatment. The diagnosis/treatment data includes, for example, data obtained in various environments such as by using apparatuses from mutually-different manufacturers, apparatuses of mutually-different versions, the same apparatus having mutually-different settings, and the like. The diagnosis/treatment data does not necessarily have to be objective data such as numerical values and may be subjective data expressed with non-numerical values such as text, for example. The diagnosis/treatment data includes, for instance, examination history information, image information, electrocardiogram information, vital sign information, medication history information, report information, medical record written information, nurse record information, referral letters, a hospital discharge summary, and the like. For example, the examination history information is information indicating a history of examination results obtained as a result of performing a specimen examination, a bacteriological examination, and the like on the patient. For example, the image information is information indicating whereabouts of medical images obtained by imaging the patient or the like. For example, the image information includes information indicating the whereabouts of a medical image file generated by the medical image diagnosis apparatus as a result of performing an examination. For example, the electrocardiogram information is information related to an electrocardiographic waveform taken from the patient. For example, the vital sign information is basic information related to vitality of the patient. For example, the vital sign information includes a pulse rate, a respiration rate, a body temperature, blood pressure, and a consciousness level. For example, the medication history information is information indicating a history of amounts of medication administered for the patient. For example, the report information is information summarizing the state and diseases of the patient, as a result of an image interpreting doctor at a radiology department interpreting medical images such as an X-ray image, a CT image, an MRI image, an ultrasound image, and/or the like, in response to an examination request received from a diagnosis/treatment doctor at a diagnosis/treatment department. For example, the report information includes image interpretation report information indicating an image interpretation report generated as a result of the image interpreting doctor referencing a medical image file stored in the PACS. For example, the report information includes information indicating the patient ID and the patient's name and birthdate of the patient corresponding to the medical image file subject to the image interpretation. For example, the medical record written information is information input to an electronic medical record by a diagnosis/treatment doctor or the like. For example, the medical record written information includes a diagnosis/treatment record at the time of hospitalization, a disease history of the patient, a medication prescription history, and the like. For example, the nurse record information is information input to an electronic medical record by a nurse or the like. The nurse record information includes a nurse record at the time of hospitalization and the like. The nurse record information may include a meal provision record during the hospitalization. Further, the diagnosis/treatment data may further include information related to accounting.

In this situation, the diagnosis assistance system 1 may include a Vendor Neutral Archive (VNA) system, in place of the HIS 50, the RIS 70, and the PACS 90. The VNA system is an integration archive system configured to unitarily manage PACSs 90 manufactured by mutually-different manufacturers and various types of diagnosis/treatment data managed by the various clinical department systems (the HIS 50 and the RIS 70). For example, the VNA system is connected to the HIS 50, the RIS 70, and the PACS 90 via an intra-hospital network such as a LAN, so as to be able to communicate with each other. In this situation, various types of information managed and saved by the VNA system do not necessarily have to be obtained from systems manufactured by mutually-different manufacturers and may be obtained from systems manufactured by mutually the same manufacturer.

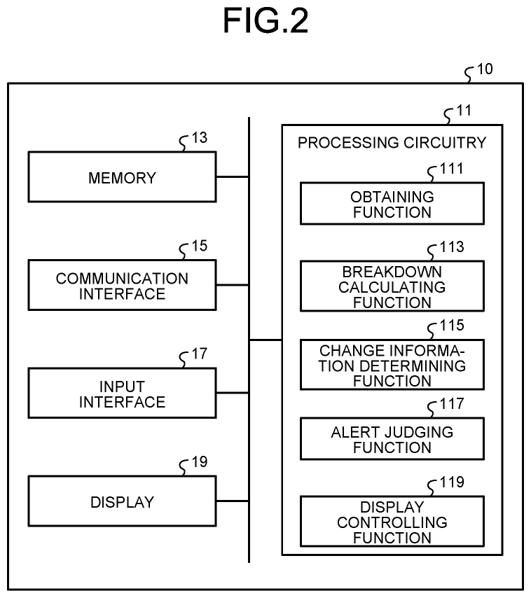

FIG. 2 is a a diagram illustrating an exemplary configuration of the diagnosis assistance apparatus 10 according to the embodiment. As illustrated in FIG. 2, the diagnosis assistance apparatus 10 includes a processing circuitry 11, a memory 13, a communication interface 15, an input interface 17, and a display 19. The processing circuitry 11, the memory 13, the communication interface 15, the input interface 17, and the display 19 are communicably connected via a bus or the like.

The memory 13 is configured to store therein various types of data. For example, the memory 13 is configured to store therein the diagnosis/treatment data received from the medical image diagnosis apparatus 30, the HIS 50, the RIS 70, and the PACS 90. Further, the memory 13 is configured to store therein information related to predetermined categories, for example. The information related to the categories will be explained later. Further, the memory 13 is configured to store therein, for example, a program for realizing the diagnosis assisting process (explained later). For example, the memory 13 is realized by using a semiconductor memory element such as a Random Access Memory (RAM) or a flash memory, or a hard disk, an optical disk, or the like. In this situation, a storage region of the memory 13 may be provided in the diagnosis assistance apparatus 10 or may be provided in an external storage device connected via a network or the like. The memory 13 is an example of a storage unit.

The communication interface 15 is configured to control transfer of various types of data and communication performed with the medical image diagnosis apparatus 30, the HIS 50, the RIS 70, and the PACS 90. For example, the communication interface 15 is configured to receive the diagnosis/treatment data from the medical image diagnosis apparatus 30, the HIS 50, the RIS 70, or the PACS 90 and to output the received diagnosis/treatment data to the processing circuitry 11. For example, the communication interface is realized by using a network card, a network adaptor, a Network Interface Controller (NIC), or the like.

The input interface 17 is configured to receive various types of input operations from the operator, to convert the received input operations into electrical signals, and to output the electrical signals to the processing circuitry 11. For example, the input interface 17 is configured to receive various types of input operations performed by the operator on various types of operation screens related to the diagnosis assisting process. In one example, the input interface 17 is configured to receive a selection from among the categories made by the operator. The categories will be explained later. In this situation, the input interface 17 is an example of an input unit.

For example, as the input interface 17, it is possible to use, as appropriate, a mouse, a keyboard, a trackball, a switch, a button, a joystick, a touchpad, a touch panel display device, and/or the like. In the present embodiments, the input interface 17 does not necessarily have to include physical operation component parts described above. Possible examples of the input interface 17 include an electrical signal processing circuitry configured, for instance, to receive an electrical signal corresponding to an input operation from an external input device provided separately from the apparatus and to output the electrical signal to the processing circuitry 11. Further, the input interface 17 may be configured by using a tablet terminal or the like capable of wirelessly communicating with the main body of the diagnosis assistance apparatus 10.

The display 19 is configured to display various types of information. For example, the display 19 is configured to output a Graphical User Interface (GUI) or the like generated by the processing circuitry 11 and used for receiving various types of operations from the operator. The GUI used for receiving the various types of operations from the operator include various types of operation screens related to the diagnosis assisting process. For example, the display 19 is configured to output a display screen related to the diagnosis assisting process and generated by the processing circuitry 11. The display screen related to the diagnosis assisting process (explained in detail later) is a display screen including at least one selected from among: diagnosis/treatment data, breakdown information of diagnosis/treatment data, change information of a patient's state, and an alert related to a patient's state. As the display 19, it is possible to use, as appropriate, arbitrary one or more of various types of display devices. For example, as the display 19, it is possible to use a Liquid Crystal Display (LCD) device, a Cathode Ray Tube (CRT) display device, an Organic Electroluminescence Display (OELD) device, or a plasma display device. The display 19 is an example of a display unit.

In this situation, the display 19 may be of a desktop type or may be structured by using a tablet terminal or the like capable of wirelessly communicating with the main body of the diagnosis assistance apparatus 10. Further, one or more projectors may be used as the display 19.

The processing circuitry 11 is configured to control operations of the entirety of the diagnosis assistance apparatus 10. As hardware resources thereof, the processing circuitry 11 includes a processor and memory elements such as a ROM and a RAM. By employing a processor configured to execute programs loaded into a memory, the processing circuitry 11 is configured to execute, among others, an obtaining function 111, a breakdown calculating function 113, a change information determining function 115, an alert judging function 117, and a display controlling function 119. In this situation, the processing circuitry 11 is an example of a processing unit. Further, the processing circuitry 11 realizing the obtaining function 111 is an example of an obtaining unit. The processing circuitry 11 realizing the breakdown calculating function 113 is an example of a breakdown calculating unit. The processing circuitry 11 realizing the change information determining function 115 is an example of a change information determining unit. The processing circuitry 11 realizing the alert judging function 117 is an example of an alert judging unit. The processing circuitry 11 realizing the display controlling function 119 is an example of a display controlling unit.

By employing the obtaining function 111, the processing circuitry 11 is configured to obtain various types of diagnosis/treatment data from the medical image diagnosis apparatus 30, the HIS 50, the RIS 70, and the PACS 90, via the network 9. In the following sections, to simplify the explanations, an example will be explained in which the obtaining function 111 obtains the medical record written information and the nurse record information, as the diagnosis/treatment data.

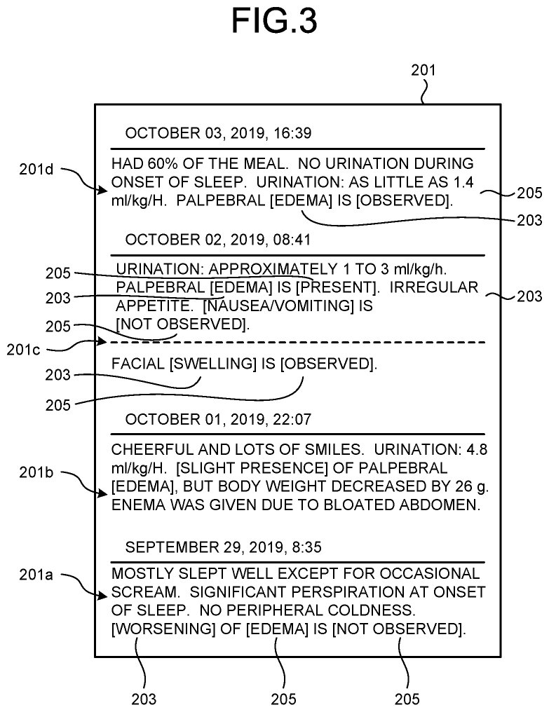

FIG. 3 is a drawing illustrating an example of diagnosis/treatment data 201 according to the embodiment. As illustrated in FIG. 3, the diagnosis/treatment data 201 includes a plurality of diagnosis/treatment record entries 201a, 201b, 201c, and 201d at a plurality of points in time related to a certain patient. Although FIG. 3 illustrates the diagnosis/treatment data 201 related to the arbitrary single patient, the obtaining function 111 according to the embodiment is configured to obtain diagnosis/treatment data 201 related to a plurality of (two or more) patients. In this situation, the patient related to the diagnosis/treatment data 201 is an example of the first patient or the second patient. Further, the plurality of diagnosis/treatment record entries 201a, 201b, 201c, and 201d are each an example of the plurality of diagnosis/treatment records at the plurality of points in time.

In the example in FIG. 3, as the diagnosis/treatment data 201, the obtaining function 111 obtains text information reading “Mostly slept well except for occasional scream. Significant perspiration at onset of sleep. No peripheral coldness. Worsening of edema is not observed.” that represents the diagnosis/treatment record entry 201a at the point in time “8:35, Sep. 29, 2019”. Further, the obtaining function 111 obtains text information reading “Cheerful and lots of smiles. Urination: 4.8 ml/kg/H. Slight presence of palpebral edema, but body weight decreased by 26 g. Enema was given due to bloated abdomen.” that represents the diagnosis/treatment record entry 201b at the point in time “22:07, Oct. 1, 2019”. Further, the obtaining function 111 obtains text information reading “Urination: approximately 1-3 ml/kg/h. Palpebral edema is present. Irregular appetite. Nausea/vomiting is not observed.” and “Facial swelling is observed” that represent the diagnosis/treatment record entry 201c at the point in time “08:41, Oct. 2, 2019”. Also, the obtaining function 111 obtains text information reading “Had 60% of the meal. No urination during onset of sleep. Urination: as little as 1.4 ml/kg/H. Palpebral edema is observed.” that represents the diagnosis/treatment record entry 201d at the point in time “16:39, Oct. 3, 2019”.

Further, the obtaining function 111 is configured to obtain information related to the categories, from the memory 13, for example. The information related to the categories include a hierarchical structure of the categories and words belonging to each of the categories.

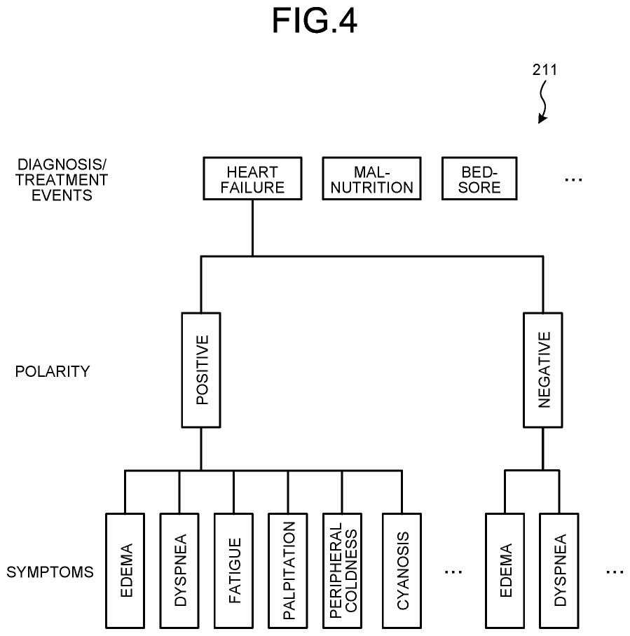

FIG. 4 is a diagram illustrating examples of categories 211 according to the embodiment. As illustrated in FIG. 4, the categories 211 have a hierarchical structure including a plurality of levels. FIG. 4 illustrates the hierarchical structure including three levels of “diagnosis/treatment events”, “polarity”, and “symptoms” starting with the highest level.

FIG. 4 illustrates “heart failure”, “malnutrition”, and “bedsore” as items at the level related to the “diagnosis/treatment events”. Further, FIG. 4 illustrates “positive” and “negative”, as items at the level related to the “polarity” of “heart failure”. Also, FIG. 4 illustrates “edema”, “dyspnea”, “fatigue”, “palpitation”, “peripheral coldness”, and “cyanosis”, as items at the level related to the “symptoms” of “heart failure” and “positive”. In addition, FIG. 4 illustrates “edema” and “dyspnea”, as items at the level related to the “symptoms” of “heart failure” and “negative”.

In this situation, it is possible to arbitrarily set the sequential order of the items at the various levels in the hierarchical structure of the categories, in accordance with a request made at the time of understanding chronological changes in the state of the patient. For example, the hierarchical structure illustrated in FIG. 4 may be used when the user wishes to understand changes in the state of the patient over multiple diagnosis/treatment events. In another example, when the user wishes to understand changes in the state of the patient with respect to an arbitrary symptom, it is possible to use a hierarchical structure in the sequential order of “diagnosis/treatment events”, “polarity”, and “symptoms”, starting with the highest level. Further, the sequential order of the items at the various levels in the hierarchical structure of the categories may dynamically be changed during the diagnosis assisting process, in accordance with an operation performed by the operator on the input interface 17.

Furthermore, the items at the various levels in the hierarchical structure of the categories are not limited to the “diagnosis/treatment events”, “polarity”, and “symptoms” illustrated in FIG. 4 and may arbitrarily be set. For example, it is possible to use a level related to “actions”, as a level in the hierarchical structure of the categories. Examples of the items at the level related to the “actions” include “administrating medication”, “observation”, and “procedure”. Further, for example, it is also acceptable to use a level related to “reporter” as a level in the hierarchical structure of the categories. Examples of the items at the level related to the “reporter” include “patient”, “family”, “doctor”, and “nurse”. Alternatively, it is also possible to use items such as “subjective” and “objective”, as items at the level related to “reporter”. In that situation, in an example, the “subjective” and the “objective” may denote the “doctor” and the “nurse”, respectively. In another example, the “subjective” and the “objective” may denote “nurse A” and “nurse B”, respectively. In yet another example, the “subjective” and the “objective” may denote the “patient” and the “family”, respectively. In yet another example, the “subjective” and the “objective” may denote the “family” and the “patient”, respectively. As explained herein, at least one of the plurality of levels in the hierarchical structure of the categories is a level related to at least one selected from among “diagnosis/treatment events”, “symptoms”, “actions”, “polarity”, and “reporter”.

Although FIG. 4 illustrates “positive” and “negative” as the items at the level related to the “polarity”, it is also possible to use other items such as “present”, “absent”, “worsened”, “improved”, or “unknown”. Further, it is also acceptable to use numerical values indicating degrees of polarities, as items at the level related to the “polarity”.

For example, it is assumed that the categories 211 are determined in advance and stored in the memory 13. However, possible embodiments are not limited to this example. For example, the categories 211 may be set as a result of being input by the operator via the input interface 17 during the diagnosis assisting process.

In the present example, the items at the various levels described above are examples of the words belonging to the categories. Further, the items at the level related to the “polarity” are also examples of words indicating polarities of the writings in the diagnosis/treatment data.

Further, as the diagnosis/treatment data, the obtaining function 111 is configured to further obtain, not only the diagnosis/treatment record entries 201a, 201b, 201c, and 201d, but also numerical value information related to each of the plurality of patients. Examples of the numerical value information related to the patients include risk scores, test values from blood tests or the like, and vital signs.

Further, the obtaining function 111 is configured to obtain input results from the operator received by the input interface 17.

By employing the breakdown calculating function 113, the processing circuitry 11 is configured to perform a natural language processing process to extract words for which the categories to belong to are determined in advance and words indicating polarities of the writings, from the diagnosis/treatment record entries 201a, 201b, 201c, and 201d. In the natural language processing process, the processing circuitry 11 does not necessarily have to extract the words indicating the polarities of the writings, i.e., the words indicating the polarities of other words in the context, from the diagnosis/treatment record entries 201a, 201b, 201c, and 201d. In one example, when the categories 211 do not include the item “polarity”, the processing circuitry 11 may perform a natural language processing process so as to extract the words for which the categories to belong to are determined in advance, from the diagnosis/treatment record entries 201a, 201b, 201c, and 201d. To the natural language processing process, a publicly-known method may be applied, as appropriate. Further, the breakdown calculating function 113 is configured to classify the diagnosis/treatment record entries 201a, 201b, 201c, and 201d into at least one category, in accordance with the extracted words.

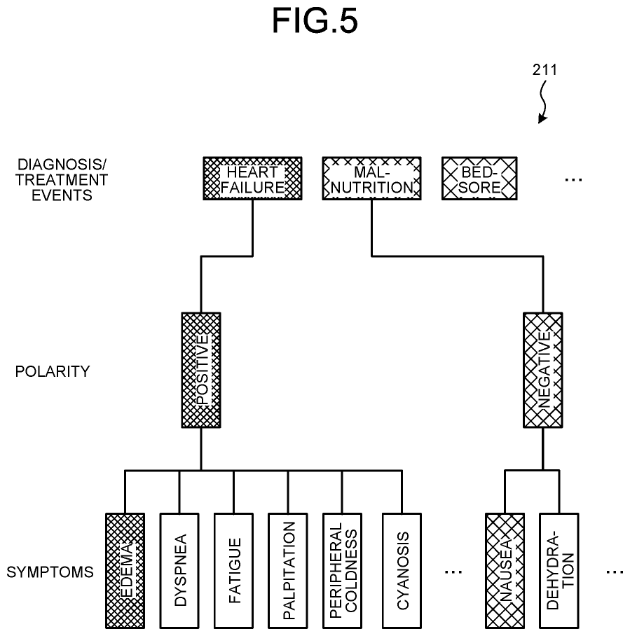

FIG. 5 is a diagram for explaining the classification of the diagnosis/treatment record entries 201a, 201b, 201c, and 201d in the diagnosis/treatment data 201 according to the embodiment. In the present example, the diagnosis/treatment record entry 201c will primarily be explained as an example. FIG. 5 illustrates “nausea” and “dehydration” as items at the levels related to “symptoms” of “malnutrition” and “negative”.

By performing a natural language processing process on the text information representing the diagnosis/treatment record entry 201c, the breakdown calculating function 113 is configured to extract “edema”, “swelling”, and “nausea/vomiting” as words 203 for which the categories to belong to are determined in advance. In this manner, in addition to the items in the hierarchical structure of the categories, the breakdown calculating function 113 extracts the words similar to the items, as the words 203 for which the categories to belong to are determined in advance. In this situation, the words similar to the items are synonyms such as words having similar meanings or paraphrasing words and are determined in advance and stored in the memory 13, for example. More specifically, it is assumed that the words similar to the items are set as words connected to nodes of the items. For example, by setting “swelling” in advance as a word similar to “edema”, the breakdown calculating function 113 is able to extract “swelling” as a word similar to “edema”, as described above. As another example, it is also possible to determine, in advance, the word “puffy” as a word similar to “edema”. Similarly, as a word similar to the item “dyspnea”, the breakdown calculating function 113 is also able to determine the word “breathlessness” in advance. Similarly, as a word similar to the item “fatigue”, the breakdown calculating function 113 is also able to determine the word “sluggishness”, in advance. As described herein, by setting the words similar to the items of the categories, it is possible to address variations of the words in the diagnosis/treatment records.

Further, by performing a natural language processing process on the text information representing the diagnosis/treatment record entry 201c, the breakdown calculating function 113 is configured to extract “present” as a word 205 indicating the polarity of the written word “edema”. Similarly, the breakdown calculating function 113 is configured to extract “observed” as another word 205 indicating the polarity of the written word “swelling”. Further, the breakdown calculating function 113 is configured to extract “not observed” as yet another word 205 indicating the polarity of the written word “nausea/vomiting”. In other words, the words 205 indicating the polarities include one or more negative words.

In this situation, as illustrated in FIG. 5, the breakdown calculating function 113 classifies the diagnosis/treatment record entry 201c into the categories of “heart failure”, “positive”, and “edema”. Further, as illustrated in FIG. 5, the breakdown calculating function 113 classifies the diagnosis/treatment record entry 201c into the categories of “malnutrition”, “negative”, and “nausea”.

Further, the breakdown calculating function 113 is configured to also classify the other diagnosis/treatment record entries into at least one category in a similar manner. For example, the breakdown calculating function 113 extracts “edema” as a word 203 from the text information representing the diagnosis/treatment record entry 201a, extracts “worsening” and “not observed” as words 205, and further classifies the record entry into the categories of “heart failure”, “negative”, and “edema”. In another example, the breakdown calculating function 113 extracts “edema” as another word 203 from the text information representing the diagnosis/treatment record entry 201b, extracts “slight presence” as another word 205, and further classifies the record entry in the categories of “heart failure”, “positive”, and “edema”. In yet another example, the breakdown calculating function 113 extracts “edema” as yet another word 203 from the text information representing the diagnosis/treatment record entry 201d, extracts “observed” as yet another word 205, and further classifies the record entry in the categories of “heart failure”, “positive”, and “edema”.

As explained above, the breakdown calculating function 113 is configured to classify the diagnosis/treatment record entries into at least one category in accordance with the words extracted from the text information representing the diagnosis/treatment record entries of each patient and the polarities of the words in the context. The polarities of the words in the context may be expressed as events expressed by the words or the polarities of the meanings. Further, the breakdown calculating function 113 is configured to calculate the breakdown information indicating the frequency of appearance of the categories related to the diagnosis/treatment records in the predetermined period of time. In this situation, the breakdown information indicates a breakdown related to the categories at mutually the same level, within a hierarchical structure such as those illustrated in FIGS. 4 and 5. For example, with respect to the “polarity”, the breakdown information is information indicating a breakdown of the number of diagnosis/treatment record entries classified in each of the categories such “positive” and “negative”. For example, with respect to the diagnosis/treatment events, the breakdown information is information indicating a breakdown of the number of diagnosis/treatment record entries classified in each of the categories such “heart failure”, “malnutrition”, and “bedsore”.

It is assumed that the time period for which the breakdown information is calculated is arbitrarily set by the operator and is stored in the memory 13, for example. The time period may be varied among diseases, diagnosis/treatment departments, or hospital wards. In one example, a shorter period of time may be set for an ICU than for a general hospital ward, for the purpose of addressing sudden changes in the states of patients. In another example, a longer period of time may be set for a recuperation ward than for a general hospital ward, for the purpose of understanding long-term changes in the states of patients.

Further, as the breakdown information, the breakdown calculating function 113 is configured to further calculate a ratio of frequency of appearance with respect to the categories related to the diagnosis/treatment records in a predetermined period of time. In one example, the breakdown calculating function 113 is configured to calculate the ratio by using a total number of all the categories at mutually the same level as a denominator and using the number of categories in question as a numerator. In this situation, when a plurality of categories are assigned to one diagnosis/treatment record entry, the total number of all the categories may be larger than the number of categories on the hierarchical level.

In another example, the breakdown calculating function 113 is configured to calculate the ratio by using a total number of diagnosis/treatment record entries as a denominator and using the number of diagnosis/treatment record entries to which the category in question is assigned as a numerator. In this situation, when a plurality of categories are assigned to one diagnosis/treatment record entry, the ratio may be calculated by narrowing down the categories to one category that represents the diagnosis/treatment record entries. Alternatively, it is also acceptable to assign a value between 0 and 1 to each of the categories with respect to the diagnosis/treatment record entries. In the example explained with reference to FIG. 5, for instance, with respect to the diagnosis/treatment record entry 201c, 0.3 and 0.7 may be assigned to “heart failure” and “malnutrition”, respectively.

By employing the change information determining function 115, the processing circuitry 11 is configured to determine the change information of the patient's state indicating a state of the patient, on the basis of a temporal change in the frequency of appearance of the categories, i.e., the breakdown information. Examples of the change information of the patient's state include “worsened”, “improved”, and “sustained”. For example, when there is a small temporal change in the frequency of appearance or the ratio of a category, the change information determining function 115 determines that the change information of the patient's state is “sustained”. As another example, when the frequency of appearance or the ratio of a category having a negative “polarity” such as “negative” has increased to exceed a predetermined threshold value, the change information determining function 115 determines that the change information of the patient's state is “worsened”. As yet another example, when the frequency of appearance or the ratio of a category having a positive “polarity” such as “positive” has increased to exceed a predetermined threshold value, the change information determining function 115 determines that the change information of the patient's state is “improved”. In the present examples, it is assumed that the predetermined threshold values are each determined in advance and stored in the memory 13. In this situation, the predetermined threshold values may be varied among diseases, diagnosis/treatment departments, or hospital wards. For example, as for the threshold value related to the frequency of appearance or the ratio of a category having a negative “polarity” such as “negative”, a smaller threshold value may be set for an ICU than for a general hospital ward, for the purpose of addressing sudden changes in the states of patients. Furthermore, the change information may be determined on the basis of temporal changes in the ratio of each category. Further, the change information may be a numerical value such as a value between 0 and 1, for example.

By employing the alert judging function 117, the processing circuitry 11 is configured to make judgement on an alert related to the patient's state indicating a state of the patient, on the basis of the breakdown information and the change information. In this situation, making the judgment on the alert denotes, for example, judging whether or not the patient is a patient for whom the user (e.g., a medical doctor) is recommended to implement observation or intervention with priority. In one example, when the frequency of appearance or the ratio has increased to exceed a predetermined threshold value with respect to a category having a negative “polarity” such as “negative”, the alert judging function 117 notifies the user (e.g., a medical doctor) that the patient is a patient for whom it is recommended to implement observation or intervention with priority, i.e., determines that an alert is to be issued. In this situation also, the predetermined threshold value may arbitrarily be set in advance and may be varied among diseases, diagnosis/treatment departments, or hospital wards. Further, the alert judging function 117 may make judgment on the alert related to the patient's state, further on the basis of numerical value information such as the vital signs obtained by the obtaining function 111. In that situation, it is possible to improve the level of precision of the alerts.

By employing the display controlling function 119, the processing circuitry 11 is configured to generate image data of a display screen including a temporal transition and an accumulated total related to the frequency of appearance of the categories, on the basis of the calculated breakdown information. Further, the display controlling function 119 causes the display 19 to display the generated image data of the display screen. The display screen will be explained later.

In the present example, the functions 111, 113, 115, 117, and 119 do not necessarily have to be realized by the single processing circuit. It is also acceptable to structure the processing circuitry 11 by combining together a plurality of independent processors, so that the functions 111, 113, 115, 117, and 119 are realized as a result of the processors executing the programs. In this situation, the functions 111, 113, 115, 117, and 119 may be realized as being distributed among or integrated into one or more processing circuits, as appropriate.

Further, although the diagnosis assistance apparatus 10 was described as being configured to execute the plurality of functions with the single computer, it is also acceptable to have the plurality of functions executed by separate computers. For example, the functions of the processing circuitry 11 such as the breakdown calculating function 113, the change information determining function 115, and the alert judging function 117 may be provided in a distributed manner.

Next, the diagnosis assisting process performed by the diagnosis assistance system 1 according to the embodiment will be explained in further detail, with reference to drawings.

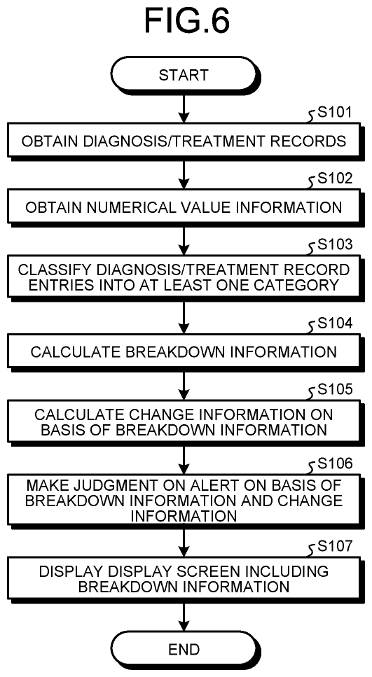

FIG. 6 is a flowchart illustrating an example of the diagnosis assisting process according to the embodiment.

The obtaining function 111 obtains text information representing diagnosis/treatment record entries, as diagnosis/treatment data (step S101). Further, the obtaining function 111 obtains numerical value information such as vital signs, as diagnosis/treatment data (step S102). The obtaining function 111 temporarily stores the obtained diagnosis/treatment data into the memory 13.

The breakdown calculating function 113 classifies the diagnosis/treatment record entries into at least one category, by performing a natural language processing process on the text information representing the diagnosis/treatment record entries (step S103). Further, the breakdown calculating function 113 calculates breakdown information indicating frequency of appearance of the categories related to the diagnosis/treatment record entries in the predetermined period of time (step S104).

Further, on the basis of breakdown information, i.e., temporal changes in the calculated frequency of appearance of the categories, the change information determining function 115 determines change information of the patient's state indicating a state of the patient (step S105). The alert judging function 117 makes judgment on an alert related to the patient's state indicating the state of the patient, on the basis of the breakdown information and the change information (step S106).

Subsequently, on the basis of the calculated breakdown information, the display controlling function 119 causes the display 19 to display a display screen including a temporal transition and an accumulated total related to the frequency of appearance of the categories (step S107). After that, the flow in FIG. 6 ends.

Alternatively, at the stage when the breakdown information has been calculated, i.e., prior to the process at step S105, the display controlling function 119 may cause the display 19 to display a display screen including the breakdown information. In that situation, the display controlling function 119 may update the display screen from time to time, in accordance with determination of the change information and judgment results on the alerts.

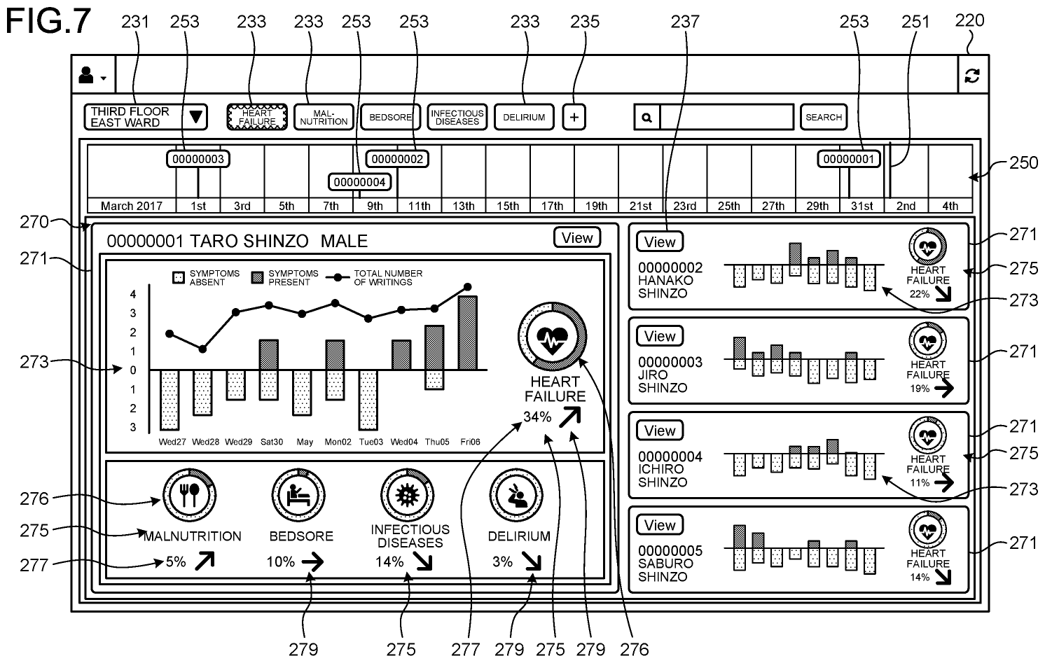

FIG. 7 is a drawing illustrating an example of a display screen 220 displayed by the diagnosis assistance system 1 according to the embodiment.

The display controlling function 119 causes the display 19 to display the display screen 220. As illustrated in FIG. 7, the display screen 220 includes a breakdown information display region 270. The breakdown information display region 270 includes display elements 271 of the breakdown information of multiple patients. Further, the display screen 220 includes icons 233 representing categories such as “heart failure”, “malnutrition”, “bedsore”, “infectious diseases”, and “delirium”. For example, by using the input interface 17, the operator selects one of the icons 233 representing a desired category. In this situation, the display controlling function 119 displays the selected icon 233 in an emphasized manner and further causes the breakdown information of the patients related to the selected category to be displayed in the breakdown information display region 270. FIG. 7 illustrates an example in which the category “heart failure” is selected.

In this situation, by selecting an icon 235 representing a category addition through the input interface 17, for example, the operator is able to have displayed another category that is not currently displayed.

For example, each of the display elements 271 includes patient information such as a patient ID and the patient's name. For example, by using the input interface 17, the operator selects one of the display elements 271 related to a patient whose details of the breakdown information the operator wishes to check. In this situation, the display controlling function 119 displays the display element 271 representing the breakdown information related to the patient selected by the operator so as to be larger than the display elements 271 representing the breakdown information related to the other patients. In this situation, in the display element 271 of the selected patient, the display controlling function 119 may additionally display the patient information thereof. FIG. 7 illustrates an example in which the gender of the patient is further displayed.

Each of the display elements 271 further includes a display element 273 indicating a temporal transition related to the frequency of appearance of the selected category and a summary display element 275. The summary display element 275 includes an icon representing the selected category, an accumulated total display element 276, a ratio display element 277, and a change information display element 279.

The temporal transition display element 273 indicates time-series data of the frequency of appearance of the diagnosis/treatment record entries that are from a predetermined period of time and have been classified in the selected category. On the display screen 220 in FIG. 7, the temporal transition display element 273 displays the number of record entries classified in “heart failure” and “symptoms are absent” and the number of record entries classified in “heart failure” and “symptoms are present” with respect to each day for the ten-day period. Further, on the display screen 220 in FIG. 7, the temporal transition display element 273 further includes a line graph indicating a total number of writings. By viewing the temporal transition display element 273, the operator is able to easily understand about the selected patient “Taro Shinzo” that symptoms (observations on his/her body) related to heart failure have been increasing since two days earlier.

On the display screen 220 in FIG. 7, the accumulated total display element 276 displays a pie graph indicating an accumulated total corresponding to the temporal transition display element 273. On the display screen 220 in FIG. 7, the ratio display element 277 indicates a percentage representing a ratio value. By viewing the accumulated total display element 276, the operator is able to intuitively understand about the selected patient “Taro Shinzo” that writings related to heart failure are increasing in electronic medical records and nurse record information. Further, by viewing the ratio display element 277, the operator is able to understand from a quantitative index that writings related to heart failure are increasing in electronic medical records and nurse record information.

On the display screen 220 in FIG. 7, the change information display elements 279 display arrow icons representing “worsened”, “improved”, and “sustained”. The change information display element 279 representing “worsened” is expressed with an arrow pointing downward. The change information display element 279 representing “improved” is expressed with an arrow pointing upward. The change information display element 279 representing “sustained” is expressed with an arrow extending horizontally. By viewing the change information display elements 279, the operator is able to intuitively understand changes in the condition of the selected patient “Taro Shinzo”.

Further, the display element 271 of the selected patient further includes summary display elements 275 related to the other categories at the same level as the selected category. On the display screen 220 in FIG. 7, the display element 271 of the selected patient “Taro Shinzo” further includes the summary display elements 275 related to the categories other than the selected “heart failure”, i.e., the categories of “malnutrition”, “bedsore”, “infectious diseases”, and “delirium”. By viewing the summary display elements 275 related to the other categories, the operator is able to check the breakdown information related to the selected patient in a view of higher perspective. In the example in FIG. 7, for instance, the operator is able to understand that there seem to be few symptoms other than those in the circulatory system.

Further, the display screen 220 includes breakdown information display elements 271 related to the patients other than the selected patient. Because the breakdown information related to the multiple patients is traversely displayed in this manner, it is possible to easily understand whether or not another patient having a high priority regarding heart failure is immediately present.

Further, as illustrated in FIG. 7, the display screen 220 further includes a timing display region 250. The timing display region 250 includes an icon 251 indicating the current point in time and alert display elements 253. The alert display elements 253 are display elements indicating when an alert was issued and for which patient. For example, from the example in FIG. 7, for instance, the operator is able to easily understand that an alert was issued approximately two days ago for the patient “Taro Shinzo” identified with the patient ID “00000001”. Further, although FIG. 7 illustrates the example in which the patient IDs are displayed in the timing display region 250, icons representing categories may further be displayed.

Further, the display screen 220 further includes an icon 231 representing a hospital ward. By selecting the icon 231 and using a pull-down menu through the input interface 17, the operator is also able to check the breakdown information related to the patients in other hospital wards. In this situation, the display controlling function 119 is also able to narrow down the patients to be displayed simultaneously by diseases, hospital wards, responsible doctors, and the like, in accordance with inputs received from the operator through the input interface 17, for example.

Further, on the display screen 220, the display controlling function 119 may display the diagnosis/treatment data itself that has been classified in a selected category. For example, by using the input interface 17, the operator may select a “View” icon 237 used for instructing to have details displayed, from within the display element 271 related to the patient whose details the operator wishes to check. In this situation, the display controlling function 119 causes the display 19 to display a display screen including the diagnosis/treatment record entries classified in the selected category. For example, when “heart failure” is the selected category, the display controlling function 119 may cause the display 19 to display a display screen including the diagnosis/treatment data 201 illustrated in FIG. 3, for example.

As explained above, the diagnosis assisting process according to the embodiment is configured to calculate the breakdown information with respect to the diagnosis/treatment data of each of the patients, by quantifying and abstracting the text information representing each of the plurality of pieces of diagnosis/treatment data related to the plurality of patients. Further, the diagnosis assisting process is configured to display the display screen including the calculated breakdown information. In other words, the diagnosis assisting process is configured to visualize the chronological changes in the states of the patients. In this situation, the quantifying and the abstracting of the diagnosis/treatment data includes, as explained above, calculating the ratios of being positive and being negative with respect to the written content of the diagnosis/treatment data. Further, the quantifying and the abstracting of the diagnosis/treatment data includes calculating a percentage of a certain topic in all the writings in the diagnosis/treatment data. Further, the quantifying and the abstracting of the diagnosis/treatment data includes calculating whether or not the abovementioned percentage is increasing in comparison to a number of days in the past. Further, the quantifying and the abstracting of the diagnosis/treatment data includes calculating how many mutually-different types of symptoms are observed, with respect to the written content of the diagnosis/treatment data. Further, the quantifying and the abstracting of the diagnosis/treatment data includes calculating how many doctors and/or nurses have reported an arbitrary topic, with respect to the written content of the diagnosis/treatment data.

The term “processor” used in the above explanations denotes, for example, a circuit such as a Central Processing Unit (CPU), a Graphics Processing Unit (GPU), an Application Specific Integrated Circuit (ASIC), or a Programmable Logic Device (PLD). Examples of the PLD include a Simple Programmable Logic Device (SPLD), a Complex Programmable Logic Device (CPLD), and a Field Programmable Gate Array (FPGA). The one or more processors are configured to realize the functions by reading and executing the programs saved in a memory. The memory having the programs saved is a non-transitory computer-readable recording medium. Alternatively, instead of having the programs saved in the memory, it is also acceptable to directly incorporate the programs in the circuits of one of more processors. In that situation, the one or more processors are configured to realize the functions by reading and executing the programs incorporated in the circuits thereof. Further, instead of executing the programs, it is also acceptable to realize the functions corresponding to the programs by using a combination of logic circuits. Furthermore, the processors of the present embodiments do not each necessarily have to be structured as a single circuit. It is also acceptable to structure one processor by combining together a plurality of independent circuits, so as to realize the functions thereof. Further, it is also acceptable to integrate two or more of the constituent elements in FIG. 1 into a single processor, so as to realize the functions thereof.

According to at least one aspect of the embodiments described above, it is possible to visualize the chronological changes in the states of the patients, on the basis of the text information of the diagnosis/treatment records.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A diagnosis assistance apparatus comprising processing circuitry configured:
to obtain text information representing each of a plurality of diagnosis/treatment records at a plurality of points in time, as diagnosis/treatment data related to a first patient;
to perform a natural language processing process to extract a predetermined word from the text information; to classify each of the plurality of diagnosis/treatment records into at least one category in accordance with the extracted word; to calculate breakdown information indicating frequency of appearance of one or more categories related to one or more diagnosis/treatment records in a predetermined period of time among the plurality of diagnosis/treatment records; and
to cause a display to display a display screen including a temporal transition and an accumulated total related to the frequency of appearance of the one or more categories on a basis of the calculated breakdown information.

2. The diagnosis assistance apparatus according to claim 1, wherein the processing circuitry further extracts a polarity of the word in a context from the text information during the natural language processing process and classifies each of the plurality of diagnosis/treatment records into said at least one category in accordance with the extracted word and the polarity.

3. The diagnosis assistance apparatus according to claim 1, wherein
the categories have a hierarchical structure including a plurality of levels, and
the breakdown information indicates a breakdown related to categories on a mutually same level in the hierarchical structure.

4. The diagnosis assistance apparatus according to claim 1, wherein
the categories have a hierarchical structure including a plurality of levels, and
at least one of the plurality of levels is a level related to one selected from among: a diagnosis/treatment event, a symptom, an action, a polarity, and a reporter.

5. The diagnosis assistance apparatus according to claim 1, wherein the processing circuitry further calculates a ratio of the one or more categories as the breakdown information and causes the display to display the display screen further including the ratio of the one or more categories.

6. The diagnosis assistance apparatus according to claim 1, wherein
the processing circuitry is further configured to determine change information of a patient's state indicating a state of the first patient on a basis of a temporal change in the frequency of appearance of the one or more categories, and
the processing circuitry causes the display to display the display screen further including the change information.

7. The diagnosis assistance apparatus according to claim 6, wherein
the processing circuitry is further configured to make judgment on an alert related to the patient's state indicating the state of the first patient on a basis of the breakdown information and the change information, and
when the alert is determined to be issued, the processing circuitry causes the display to display the display screen further including the alert related to the patient's state.

8. The diagnosis assistance apparatus according to claim 7, wherein
the processing circuitry further obtains numerical value information of the first patient as the diagnosis/treatment data related to the first patient, and
the processing circuitry makes the judgment on the alert related to the patient's state further on a basis of the numerical value information.

9. The diagnosis assistance apparatus according to claim 1, wherein
the processing circuitry obtains the text information related to a second patient different from the first patient,
the processing circuitry further calculates the breakdown information with respect to the second patient, and
the processing circuitry causes the display to display a display screen including the temporal transition and the accumulated total related to each of the first and the second patients.

10. The diagnosis assistance apparatus according to claim 1, further comprising: an input interface configured to receive a selection from among the categories made by an operator, wherein
the processing circuitry causes the display to display a display screen including one of the plurality of diagnosis/treatment records classified into the selected category.

11. The diagnosis assistance apparatus according to claim 1, further comprising: an input interface configured to receive a selection from among the categories made by an operator, wherein
the processing circuitry causes the display to display a display screen including the temporal transition and the accumulated total related to the selected category.

12. A diagnosis assistance system comprising:
processing circuitry configured to obtain text information representing each of a plurality of diagnosis/treatment records at a plurality of points in time as diagnosis/treatment data related to a first patient, to perform a natural language processing process to extract a predetermined word from the text information, to classify each of the plurality of diagnosis/treatment records into at least one category in accordance with the extracted word, and to calculate breakdown information indicating frequency of appearance of one or more categories related to one or more diagnosis/treatment records in a predetermined period of time among the plurality of diagnosis/treatment records; and
a display configured to display a display screen including a temporal transition and an accumulated total related to the frequency of appearance of the one or more categories on a basis of the calculated breakdown information.
